(19) United States
(12) Patent Application Publication
(10) Pub. No.: US 2013/0281920 A1
(43) Pub. Date: Oct. 24, 2013

(54) Endometrial Ablation

(71) Applicants: Daniel Hawkins, Bellevue, WA (US); Nathan Kundtz, Kirkland, WA (US); John Mathew Adams, Snohomish, WA (US)

(72) Inventors: Daniel Hawkins, Bellevue, WA (US); Nathan Kundtz, Kirkland, WA (US); John Mathew Adams, Snohomish, WA (US)

(73) Assignee: Elwha LLC, a limited liability company of the State of Delaware

(21) Appl. No.: 13/451,890

(22) Filed: Apr. 20, 2012

(51) Int. Cl. A61B 18/18; A61M 13/00

(52) U.S. Cl. 604/26; 606/33

(57) ABSTRACT

A tissue ablation system includes a waveguide configured to leak microwave radiation through an array of subwavelength apertures.

### BACKGROUND

A common therapy for treatment of menorrhagia (excessive menstrual bleeding) is ablating the endometrial lining that is responsible for the bleeding. Such ablation has been shown to reduce, and in some instances, to cease the menstrual bleeding.

### SUMMARY

In one aspect, a medical device configured to be coupled to a microwave source having an operating frequency includes a radiation-confining structure (e.g., a waveguide) configured for insertion into a body cavity or lumen (e.g., a uterus), and a conductive layer surrounding the radiation-confining structure. The conductive layer includes a plurality of subwavelength apertures, which are configured to collectively produce a microwave field corresponding to a selected ablation region. The radiation-confining structure may be configured to expand within the body cavity or lumen, for example in a fan configuration. The apertures may be arranged to produce a microwave field in a shape substantially similar to the anatomical shape of the body cavity or lumen. The selected ablation region may be configured to preferentially ablate tissue in one or more desired regions, and may further be configured to spare tissue in one or more undesired regions. The radiation-confining structure may be at least partially enclosed in a shell, which may be configured not to stick to ablated tissue. The shell may be configured to cover at least a portion of the plurality of apertures. The device may further include a vacuum source configured to evacuate the body cavity or lumen, for example by removing water, steam, or smoke. The device may be configured to monitor material removed by the vacuum source to detect perforation of an organ, or the medical device may include a pressure source configured to insufflate the body cavity or lumen. The device may be configured to shut off in response to a signal condition (e.g., temperature, moisture, airflow, impedance, reflection, acoustic response, pressure, time, or rate of change of any of the above). The subwavelength apertures may also have subwavelength spacing.

In another aspect, a method of ablating tissue includes inserting a radiation-confining structure into a body cavity or lumen. The radiation-confining structure is surrounded by a conductive layer including a plurality of subwavelength apertures. The method further includes coupling a microwave source to the radiation-confining structure, whereupon the plurality of subwavelength apertures produces a microwave field corresponding to a selected ablation region. The method may further include expanding the radiation-confining structure within the body cavity or lumen. The selected ablation region may be substantially similar in shape to the body cavity or lumen, and may include a region of greater or lesser penetration. The method may further include monitoring a parameter of the body cavity or lumen (e.g., temperature, moisture, airflow, impedance, reflection, acoustic response, pressure, time, or rate of change of any of the above) and adjusting the ablation region in response to the monitored parameter. Adjusting the ablation region may include increasing, decreasing, or terminating the field, and may include interposing a shield over at least a portion of the apertures. The method may further include evacuating the body cavity or lumen, for example including removing water, steam, or smoke, and may further include monitoring evacuated material.

In another aspect, a system for tissue ablation includes a radiation-confining structure, a conductive layer configured to leak microwave radiation according to a surgical plan, and a microwave source configured to be optically coupled to the radiation-confining structure. The surgical plan may include ablating tissue of a body cavity or lumen, in which case the radiation-confining structure may be configured to expand within the body cavity or lumen (e.g., in a fan configuration). The apertures may be arranged to produce the microwave field in a shape substantially similar to the anatomical shape of a body cavity or lumen. The surgical plan may include ablating tissue in one or more desired regions, and may include sparing tissue in one or more undesired regions. The radiation-confining structure may be at least partially enclosed in a shell, which may be configured not to stick to ablated tissue, and which may cover at least a portion of the conductive layer. The system may further include a vacuum source configured to evacuate a body cavity or lumen, for example including removing water, steam, or smoke. The vacuum source may be configured to monitor removed material. The system may be configured to shut off in response to a signal condition (e.g., temperature, moisture, airflow, impedance, reflection, acoustic response, pressure, time, or rate of change of any of the above).

In another aspect, a method of ablating tissue includes directing microwave radiation into a radiation-confining structure disposed in a body cavity or lumen, and leaking the radiation through subwavelength apertures in a conductive layer, wherein the leaked radiation has the effect of ablating surrounding tissue. The method may further include expanding the radiation-confining structure within the body cavity or lumen. Leaking the radiation may include leaking a greater intensity of radiation in at least one tissue region. The method may further include monitoring a parameter of the body cavity (e.g., temperature, moisture, airflow, impedance, reflection, acoustic response, pressure, time, or rate of change of any of the above) and adjusting a quantity of radiation in response to the monitored parameter. Adjusting the quantity of radiation may include increasing, decreasing, or terminating the radiation, and may include interposing a shield over at least a portion of the apertures. The method may further include evacuating the body cavity or lumen, for example including removing water, steam, or smoke, and may include monitoring removed material.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of an endometrial ablation system.

FIG. 2 is a schematic of another endometrial ablation system.

FIG. 3 is a schematic of the endometrial ablation system of FIG. 2, illustrating deployment in a uterus.

FIG. 4 is a schematic of an ablation system.

FIG. 5 is a schematic of an intestinal ablation system.

FIG. 6 shows a method of operating an endometrial ablation system.

FIG. 7 shows a method of making an endometrial ablation system.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

As used herein, “radiation-confining structure” includes waveguides and other materials that are at least partially transmissive to radiation.

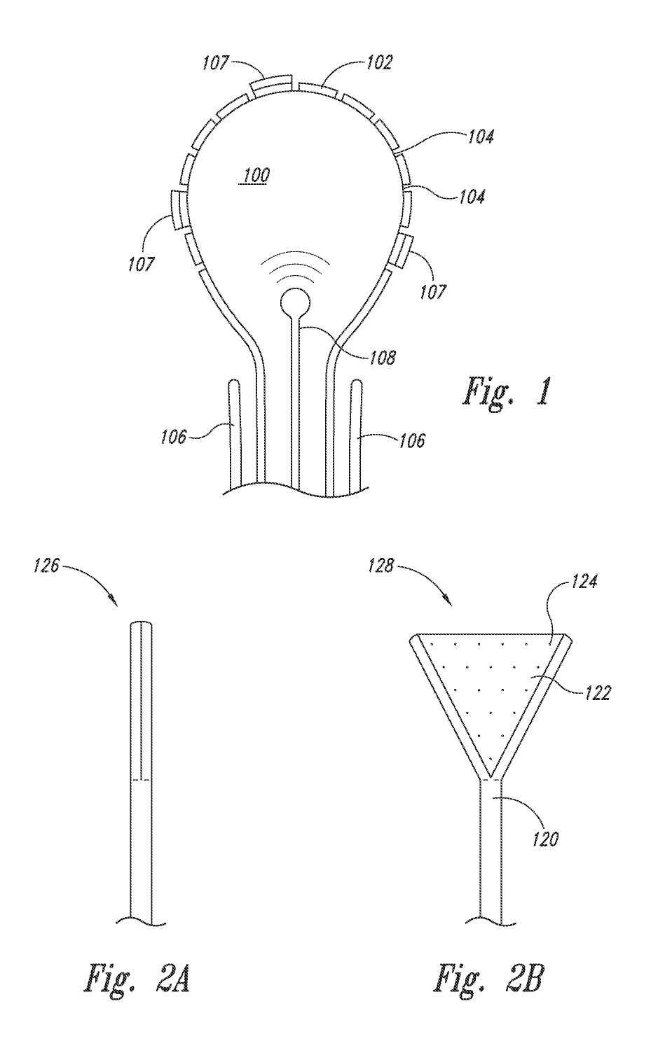

FIG. 1 illustrates one embodiment of a system for ablating a uterine lining The system includes an inflatable balloon 100, which has a thin coating of a conductive material 102 (e.g., silver, copper, or another conductive metal). The coating is perforated with a plurality of subwavelength apertures 104, which cooperate to produce a shaped field in response to a microwave input. The apertures 104 may be evenly spaced, or they may be concentrated in certain areas of the balloon 100. For example, the apertures may be placed to produce a field that penetrates more deeply in regions of the uterus where the lining is expected to be relatively thick, and more shallowly in regions where the lining is thin. Apertures 104 may be subwavelength in diameter, in spacing, or in both.

Radiation through a subwavelength aperture spreads diffractively. In an array of subwavelength apertures (or apertures with subwavelength spacings), the conductive surface acts as a ground plane, shaping the propagation of the microwave field. Surface antennas are discussed extensively in copending U.S. patent application Ser. No. 13/317,338, filed Oct. 14, 2011, attorney docket 0209-011-001-000000, entitled “Surface Scattering Antennas,” which is incorporated by reference herein. In this context, the subwavelength apertures act cooperatively to reshape the microwave field in the vicinity of the conductor.

In some embodiments, rather than subwavelength apertures, the coating may include active metamaterial components such as split ring resonators, whose properties can be adjusted through the use of liquid crystal, ferroelectrics, PIN diodes, varactors, or other active microwave components. Such components are described in U.S. patent application Ser. No. 13/317,338, referenced above. For a disposable balloon, apertures are generally preferred because of their low cost, but if the balloon is to be reused or there are other factors that allow a more expensive balloon, then active components may be preferred. In one such embodiment, a tunable dielectric such as liquid crystal can be used to change the properties of “apertures” such that they may be turned on and off independently or in groups.

In use, the balloon 100 is placed in the uterus and inflated to produce a contact between the coating and the uterus. A microwave source (not shown) is then coupled to coating 102, either through the balloon material or through an inserted antenna 108. Since the coating is in contact with the uterus, the uterus ablates relatively rapidly and evenly. Once the uterine lining has been sufficiently ablated, the balloon may be uninflated and withdrawn. In some embodiments, the balloon may include a “slick” or nonadhesive outermost layer so that it does not stick to the uterus. There may also be an occlusive “sleeve” 106 which may be deployed to fully or partially cover the balloon to impede the transmission of microwaves into tissue.

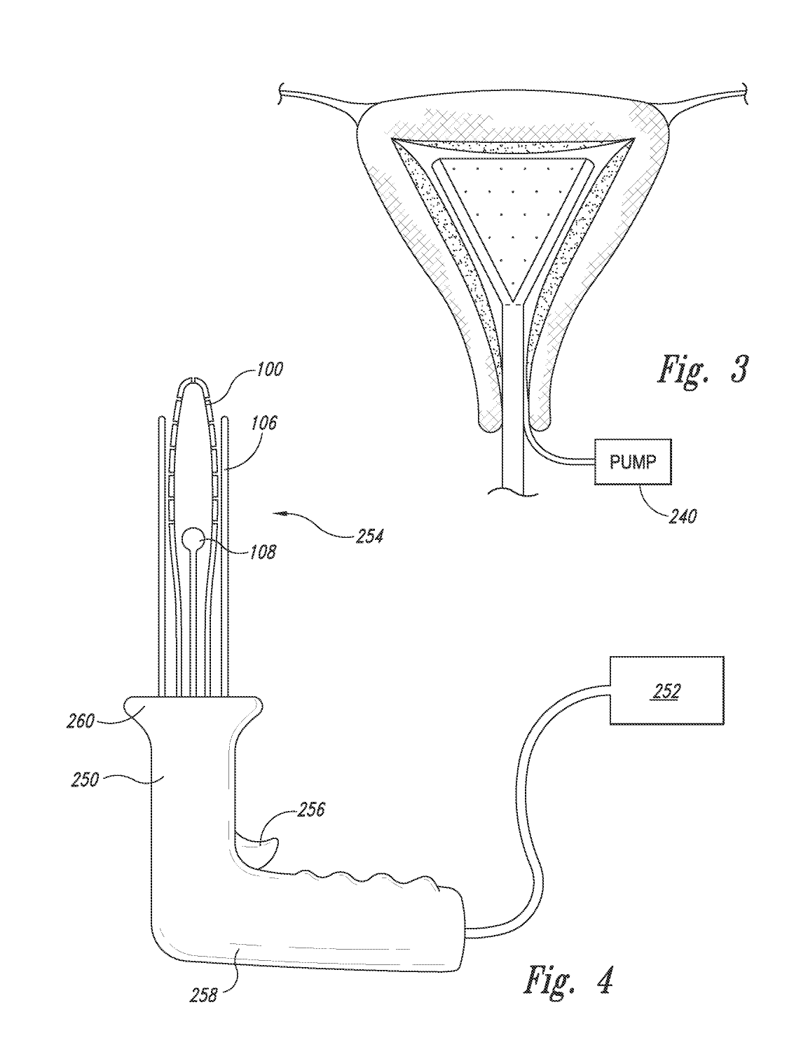

Microwaves may be delivered to the uterus for a predetermined time, or other sensing methods may be used to gauge the amount of ablation. For example, a moisture sensor 107 may measure the amount of moisture expelled by the tissue during ablation. As moisture production slows, it may be inferred that all of the tissue has been adequately ablated and the device may be turned off and withdrawn. In such embodiments, the moisture sensor may be inserted into the uterus as shown in FIG. 1, or fluid may be withdrawn from the uterus (e.g., by a vacuum system such as that shown in FIG. 3). Other sensors may also monitor moisture removal: for example, as the water content of the surrounding tissue is reduced, the absorption of that tissue will diminish. This reduced absorption may be detected as additional microwave energy being reflected from the device. This reflected signal may be used to monitor the ablation process (in addition to or as an alternative to a direct moisture sensor). Other sensors may also be used to gauge degree of ablation, such as temperature (at a specific location or at a plurality of locations, such as those shown in FIG. 1), heat input, acoustic signature, reflection (for example, using a different wavelength than the one used to treat), tissue impedance, pressure, smoke or steam, or pain. In any such embodiments, the device may be turned off by the operator (or the patient) in response to the sensor, or it may automatically shut off when a threshold is reached on the sensor.

During ablation, temperature is expected to bear a strong relationship to the quantity of moisture in tissue. See, for example, U.S. Pat. No. 6,813,520, which is incorporated by reference herein, which discusses moisture removal during an RF tissue ablation process (see particularly column 11). This fact can be used to monitor both the progress and the evenness of ablation. Thermocouples or thermistors may be positioned at one or more positions around the balloon to monitor the tissue temperature. Once temperature begins to rise above a threshold at a particular location, it may be inferred that moisture is substantially eliminated at that point and ablation at that location is substantially complete. Tissue impedance may also be monitored as a way of monitoring the amount of moisture in the tissue.

In some embodiments, the coating is divided into sections with independent inputs. In such embodiments, the signal to a particular segment of the balloon may be turned off (automatically or manually) once a temperature reading indicates that that section of tissue has been sufficiently ablated.

Reflection (for example, of visible light) and/or acoustic signature on ultrasound may also be used to monitor ablation. Ultrasound has the advantage that a transducer may be placed outside the patient, on the abdomen, to provide an independent monitor. Smoke or steam also is an indicator that ablation is complete, and is convenient to view in embodiments where a vacuum is applied (but may also be monitored within the uterus if preferred). Significant pain is typically an indication that tissue beyond the targeted tissue has been ablated, and the system should be switched off to prevent further burning.

While the particular type of microwaves applied will depend upon the clinical picture, it is expected that frequencies in the range of about 500 MHz-100 GHz will provide effective ablation. In particular, frequencies of about 2.4 GHz or about 22 GHz will excite liquid water molecules and are effective in heating tissue to ablation temperatures.

In some embodiments, aperture shapes and or spacings may be arranged to engineer coupling strength of radiation to tissue. As discussed above, in some embodiments, apertures may be spaced more closely along the sides and top of the balloon, where the endometrium is thickest, and more widely in the vicinity of the os and the fallopian tubes, where the lining is thinner. Furthermore, coupling strengths may differ with differing microwave frequencies. In such embodiments, the frequency of microwave radiation may be adjusted, for example in response to a sensor, in order to shift the ablation pattern.

FIG. 2 illustrates another embodiment for uterine ablation. In this embodiment, a radiation-transmissive material 120 (e.g., a waveguide) is coated on both sides with a conductive material 122 containing apertures 124, and fan-folded (or otherwise folded for insertion). A physically similar device (but with conventional electrodes) may be seen in U.S. Pat. No. 6,929,642, which is incorporated by reference herein. The folded waveguide 126 may be inserted through the cervix and then unfolded 128. Rather than inflating a balloon to bring it into contact with the uterus, the uterine cavity is evacuated to bring the uterus into contact with the waveguide. FIG. 3 shows the expanded device deployed in a uterus, with a pump to bring the uterine walls into contact with the device. A microwave generator (not shown) is coupled to the waveguide to provide ablative energy.

Since this embodiment is used with a vacuum pump, it is particularly well-suited to use moisture sensing to trigger the decision that ablation is complete. U.S. Pat. Nos. 6,813,520 and 7,604,633, which are incorporated by reference herein, describe alternate ablation systems that rely on moisture transport and discuss the expulsion of moisture during different stages of ablation.

The vacuum pump of FIG. 3 may also be used in some embodiments to detect perforation of the uterus. During ablation, the pump is expected primarily to draw fluid from the uterus. If it begins to draw a significant quantity of air, especially if this happens suddenly, it can be inferred that the uterus has been perforated and air is being drawn from the peritoneal cavity. In this case, the device is deactivated and the doctor may immediately treat the perforation. In some embodiments, the fluid withdrawn from the uterus may also be analyzed to ensure that peritoneal fluid is not present. In some embodiments, rather than (or in addition to) using a vacuum pump, the uterus may be insufflated at the end of the procedure to confirm that it can hold pressure. If not, it may have been perforated and treatment can be applied.

FIG. 4 illustrates the ablation element of FIG. 1 connected to a microwave source and ready for deployment. Occlusive sleeve 106 surrounds balloon 100, which contains antenna 108. All of these components are designed to plug into a housing 250, which is connected to microwave source 252. The operator may then dilate the cervix (if necessary) and insert the balloon assembly 254. Occlusive sleeve 106 is retracted, and the balloon 100 is inflated to contact the uterus. Microwave energy is coupled to the balloon by pressing trigger 256. In the illustrated embodiment, housing 250 is shaped to include handle 258, but any configuration that is comfortable for operator and patient may be used. The illustrated embodiment also includes a stop 260 that is used to ensure that the device is not inserted too far into the cervix. In some embodiments, the doctor may first use a sounding device (or any other suitable method, such as ultrasound) to determine the length of the uterus, and then adjust the position of the stop 260 to accommodate the patient's anatomy. In other embodiments, the stop 260 may be positioned at a fixed distance from the balloon 100, where the total length of the device is selected to be appropriate for the anatomy of most patients.

FIG. 5 illustrates an embodiment 300 suitable for ablation of other tissue. For example, this embodiment may be used to irradiate the small intestine. Other embodiments may be used in other body cavities or lumens, such as the urethra (e.g., for treatment of the prostate), the sinus cavities, the esophagus, or the colon. Each of these embodiments will require a somewhat differently shaped and sized instrument, but all will work according to similar principles—an expandable waveguide (or other material) 302 supports microwave radiation, which then leaks through apertures 306 in conductive coating 304. As shown in FIG. 5, the device is inflated to conform to a section of the small intestine 308. A similar device may also be effective during surgery, particularly on vascularized organs such as the lung or the liver. In these embodiments, the surfaces may be ablated to reduce bleeding during the surgery.

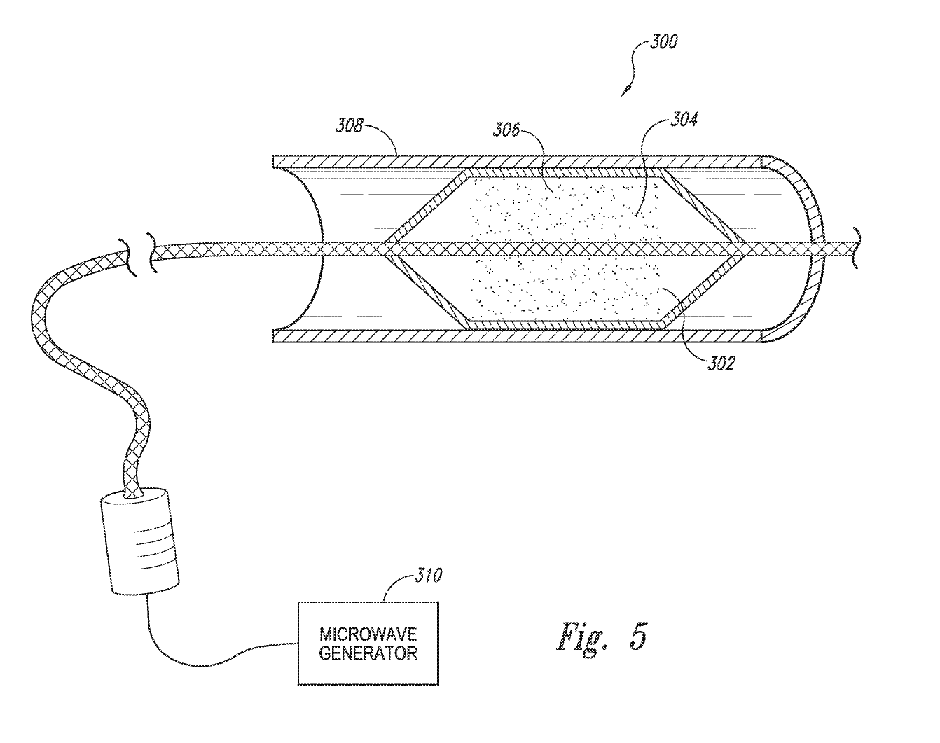

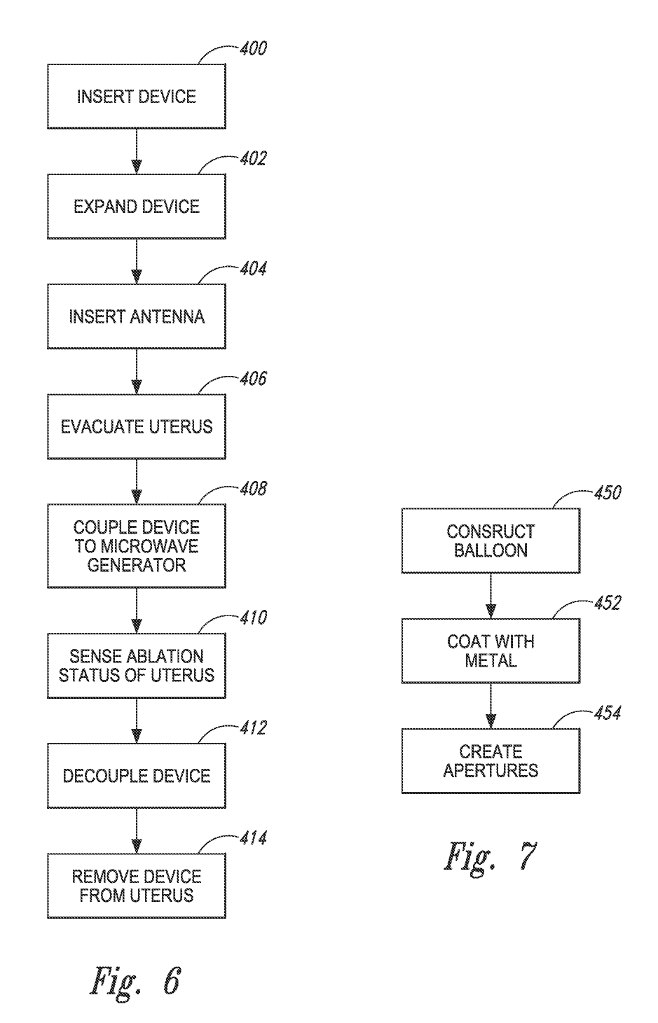

FIG. 6 shows a method of operating an ablation device such as that illustrated in FIG. 1 or FIG. 2. Although the steps of the method are shown in one potential sequence, it will be understood that in some embodiments, these steps may be carried out in a different sequence. Initially, the device is inserted into a uterus 400, and if required, it is expanded 402. If a separate antenna is used, it is also inserted 404 (in some embodiments, the antenna may be built into the balloon, or the balloon itself may act as an antenna). In some embodiments, the uterus may also be evacuated 406, but this step is optional. The device may be coupled to the microwave generator 408 before or after insertion, but in typical embodiments, the microwave generator is not turned on until the device is deployed in the uterus. In embodiments that include a sensor, the ablation of the uterus may be monitored 410, but this step is optional. Once ablation is complete, the device is decoupled 412 (or the generator is turned off), and the device is removed from the uterus 414.

FIG. 7 shows a method of making an ablation device. It will be understood that the details of making the device will be dependent upon the conditions in which it is to be used, and that this figure illustrates only a single embodiment. Balloon 100 is constructed 450, typically from a compliant plastic material suitable for use in the body. In some embodiments, the balloon 100 may be constructed from an elastomer, while in others, it may be a non-elastomeric polymer, or other flexible material such as pigskin.

The balloon 100 is then coated with a metal layer 452. A variety of coating methods may be used, as long as they result in a metal layer which is thin relative to the length of the device and which may be perforated. For example, metal may be sputtered onto the balloon in its expanded configuration. Sputtering is a particularly suitable method because it forms a thin coating that may already have sufficient subwavelength apertures. The balloon may also be coated, for example, by physical vapor deposition, chemical vapor deposition, rolling, electroplating, or electroless deposition. In any of these embodiments, once the balloon is coated, if it does not already feature apertures, they may be added 454, for example by an etching technique such as wet etching, RIE etching, ion beam etching, or laser ablation. The lumen through which the balloon will be inflated may also include a conductive channel sufficient to couple the coating 102 to the microwave generator. This channel may be created at the same time that the balloon is coated, or separately. Alternatively, if the balloon itself functions as a waveguide material, then a conductive channel may not be needed on the lumen.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A medical device configured to be coupled to a microwave source having an operating frequency, comprising:
a radiation-confining structure configured for insertion into a body cavity or lumen; and
a conductive layer surrounding the radiation-confining structure, the conductive layer having a plurality of subwavelength apertures, wherein the plurality of subwavelength apertures is configured to collectively produce a microwave field corresponding to a selected ablation region.

2. The device of claim 1, wherein the radiation-confining structure is configured to expand within the body cavity or lumen.

3. The device of claim 2, wherein the radiation-confining structure has a fan configuration.

4. The device of claim 1, wherein the body cavity or lumen is a uterus.

5. The device of claim 1, wherein the apertures are arranged to produce the microwave field in a shape substantially similar to the anatomical shape of the body cavity or lumen.

6. The device of claim 1, wherein the selected ablation region is configured to preferentially ablate tissue in one or more desired regions.

7. The device of claim 6, wherein the selected ablation region is further configured to spare tissue in one or more undesired regions.

8. The device of claim 1, wherein the radiation-confining structure is at least partially enclosed in a shell.

9. The device of claim 8, wherein the shell is configured not to stick to ablated tissue.

10. The device of claim 8, wherein the shell is configured to cover at least a portion of the plurality of apertures.

11. The device of claim 1, further comprising a vacuum source configured to evacuate the body cavity or lumen.

12. The device of claim 11, wherein the vacuum source is configured to remove water or steam from the body cavity or lumen.

13. The device of claim 11, wherein the vacuum source is configured to remove smoke from the body cavity or lumen.

14. The device of claim 11, wherein the vacuum source is configured to monitor removed material in order to detect perforation of an organ.

15. The device of claim 11, wherein the vacuum source is configured to warn the operator if it begins to draw air.

16. The device of claim 1, further comprising a pressure source configured to insufflate the body cavity or lumen.

17. The device of claim 1, wherein the device is configured to shut off in response to a signal condition.

18. The device of claim 17, wherein the signal condition is selected from the group consisting of temperature, moisture, airflow, impedance, reflection, acoustic response, pressure, time, and rate of change of any of the above.

19. The device of claim 1, wherein the subwavelength apertures have subwavelength spacing.

20. A method of ablating tissue, comprising:
inserting a radiation-confining structure into a body cavity or lumen, the radiation-confining structure surrounded by a conductive layer including a plurality of subwavelength apertures; and
coupling a microwave source to the radiation-confining structure, wherein the plurality of subwavelength apertures produces a microwave field corresponding to a selected ablation region.

21. The method of claim 20, further comprising expanding the radiation-confining structure within the body cavity or lumen.

22. The method of claim 20, wherein the selected ablation region is substantially similar in shape to the body cavity or lumen.

23. The method of claim 20, wherein the selected ablation region includes at least one region of greater penetration.

24. The method of claim 23, wherein the selected ablation region includes at least one region of lesser penetration.

25. The method of claim 20, further comprising monitoring a parameter of the body cavity or lumen and adjusting the ablation region in response to the monitored parameter.

26. The method of claim 25, wherein the monitored parameter is selected from the group consisting of temperature, moisture, airflow, impedance, reflection, acoustic response, pressure, time, and rate of change of any of the above.

27. The method of claim 25, wherein adjusting the ablation region includes increasing the field.

28. The method of claim 25, wherein adjusting the ablation region includes decreasing the field.

29. The method of claim 25, wherein adjusting the ablation region includes terminating the field.

30. The method of claim 25, wherein adjusting the ablation region includes interposing a shield over at least a portion of the apertures.

31. The method of claim 20, further comprising evacuating the body cavity or lumen.

32. The method of claim 31, wherein evacuating the body cavity or lumen includes removing water or steam.

33. The method of claim 31, wherein evacuating the body cavity or lumen includes removing smoke.

34. The method of claim 31, wherein evacuating the body cavity or lumen includes monitoring evacuated material.

35. A system for tissue ablation, comprising:
a radiation-confining structure;
a conductive layer configured to leak microwave radiation according to a surgical plan; and
a microwave source configured to be optically coupled to the radiation-confining structure.

36.-50. (canceled)

51. A method of ablating tissue, comprising:
directing microwave radiation into a radiation-confining structure disposed in a body cavity or lumen; and
leaking the radiation through subwavelength apertures in a conductive layer, wherein the leaked radiation has the effect of ablating surrounding tissue.

52.-63. (canceled)
